(19) United States
(12) Patent Application Publication
(10) Pub. No.: US 2009/0062737 A1
(43) Pub. Date: Mar. 5, 2009

(54) Ice coated hypodermic needle

(76) Inventor: William Y. Sun, Alexandria, VA (US)

Correspondence Address: CLYDE I. COUGHENOUR, 16607 SUTTON PLACE, WOODBRIDGE, VA, 22191, US

(21) Appl. No.: 12/222,885

(22) Filed: Aug. 19, 2008

Related U.S. Application Data
(63) Continuation-in-part of application No. 11/846,166, filed on Aug. 28, 2007.

(51) Int. Cl. A61M 5/42

(52) U.S. Cl. 604/112

(57) ABSTRACT

An ice coating is placed on the hypodermic needle to cool the flesh during insertion. The ice coated front end is sharp, for penetration. The ice coating can be formed by cooling the needle and dipping it in water or molding ice onto the needle. A refrigeration chamber or cryogenic chamber can be used to cool the needle. The mold for forming an ice coating can be cooled by a cryogenic fluid or refrigerant being conducted into coils of the mold surrounding heat conducting mold walls that are used to form the ice coating on the needle. The water can be sterilized with disinfectant included.

This application is a continuation-in-part of your applicant's prior patent application, Ser. No. 11/846166, filed Aug. 27, 2007, to an Ice Tip Hypodermic Needle.

### BACKGROUND OF THE INVENTION

1. Field of the Invention

The invention is to a frozen water coated hypodermic needle to make an injection reasonably pain free.

2. Description of Related Art

The use of cold surfaces and cryogenic fluids to cool the skin where injection or cutting is to take place per se is old and obvious. Examples of prior art patents that use various means to cool the skin or needle include: U.S. Pat. No. 2,536,001, issued Dec. 26, 1950, to W. Chase; U.S. Pat. No. 2,746,264, issued May 22, 1956, to F. G. Keyes; U.S. Pat. No. 3,333,587, issued Aug. 1, 1967 to R. D. Johnston; U.S. Pat. No. 3,351,063, issued Nov. 7, 1967, to Malaker et al; U.S. Pat. No. 3,399,675, issued Sep. 3, 1968, to C. W. Hill; U.S. Pat. No. 3,483,869, issued Dec. 16, 1969, to M. J. Hayhurst; U.S. Pat. No. 3,563,239, issued Feb. 16, 1971, to C. W. Hill; U.S. Pat. No. 3,605,742, issued Sep. 20, 1971, to R. C. Tibbs; U.S. Pat. No. 4,646,735, issued Mar. 3, 1987, to J. S. Seney; U.S. Pat. No. 4,725,265, issued Feb. 16, 1988, to M. Sairenji; U.S. Pat. No. 4,802,475, issued Feb. 7, 1989, to A. Weshahy; U.S. Pat. No. 5,236,419, issued Aug. 17, 1993, to J. S. Seney; U.S. Pat. No. 6,936,028, issued Aug. 30, 2005, to Hommann et al; U.S. Pat. No. 7,097,641, issued Aug. 29, 2006, to Arless et al.

### SUMMARY OF THE INVENTION

A hypodermic needle is provided with an ice coating for cooling the skin when penetrating the skin of a patient. On exposing the needle to the warmth of the atmosphere and/or body of a patient, the coating begins to melt and cool the skin permitting the injection of a fluid through the cannula of a hypodermic needle. The coating can be pure water, sterilized water or water containing a disinfectant. The coating can be frozen onto the needle at the time of manufacture or at a convenient time before use or the needle can be cryogenically cooled at the time of use and dipped in the coating fluid to have the coating frozen onto the needle just before it is used. The water used can be pure sterilized water or water that is treated with a disinfectant or purifier.

### BRIEF DESCRIPTION OF THE DRAWINGS

FIG. 1 is a side cross-section view of a hypodermic needle having an ice coating.

FIG. 2 is a cross section side view of a water holder for dipping a cold hypodermic needle.

FIG. 3 is a side cross-section view of a refrigerant cooling container for a hypodermic needle.

FIG. 4 is a cross-section side view of a cryogenic cooling container for a hypodermic needle.

FIG. 5 is a cross section of a freezer container for forming an ice coating on a hypodermic needle.

FIG. 6 is a top view of an elastic bladder showing a slit valve.

### DESCRIPTION OF THE PREFERRED EMBODIMENTS

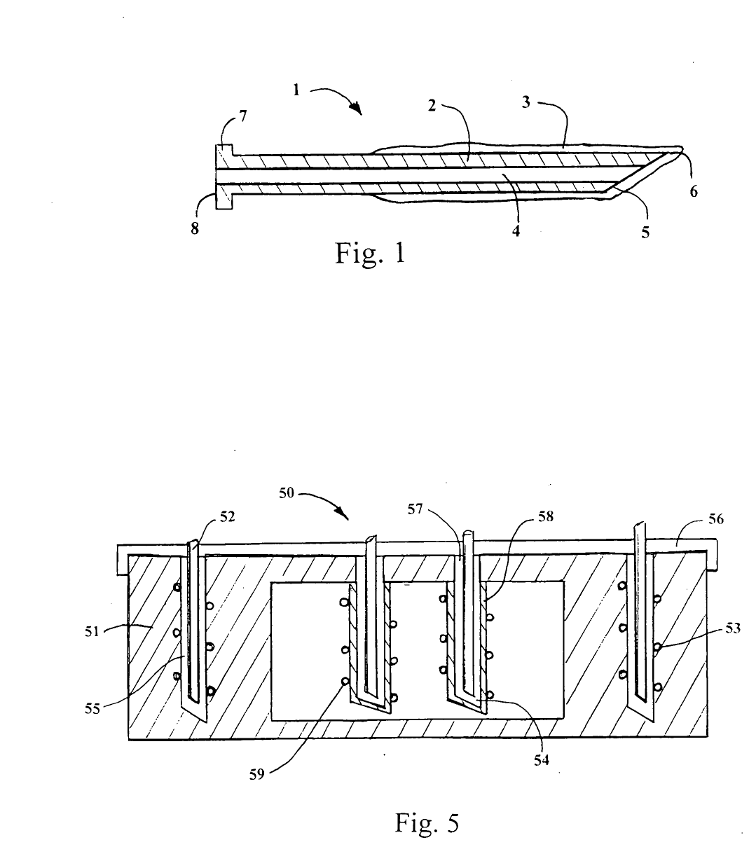

The invention is to an ice coated hypodermic needle to make an injection as painless as possible. The needle to be used is an ice coated standard needle structure. The ice coated cannula or needle 1 of a hypodermic syringe is shown in FIG. 1. A cannula shank 2 has an ice coating 3 on the front or distal or tip end 5 having a sharp needle tip and a syringe attachment or hub 7 on the proximal or rear end 8. The preferred ice coating can be from 0.010 to 0.125 inches thick.

It is known that a person suffers less or is not as sensitive to hypodermic needles where the skin or flesh surface is cold. The ice needle tip serves to cool the skin to reduce both pain and apprehension. Since the atmospheric temperature and body temperatures are both higher than the ice, as soon as the ice on the hypodermic tip end having a sharp needle tip 5 is exposed it begins to melt on its surface. The wet needle tip performs the function of assisting heat transfer from the flesh surface to the ice coating 3 and acting as a lubricant. The ice coated cannula 1 is inserted using the sharp needle tip front end 5 having a sharp flesh-penetrating point 6. The ice coating is thin and quickly melts when exposed to room temperature or inserted into the body. With the ice on the end melted, fluid can be injected through the hollow passage 4 of the hypodermic needle or cannula shank 2 into the body or body part.

The ice coated needle 2 can be “factory manufactured” with the cannula shank 2 and ice coating 3 formed together for both shipment and use or they can be placed together, for example, at the point of use.

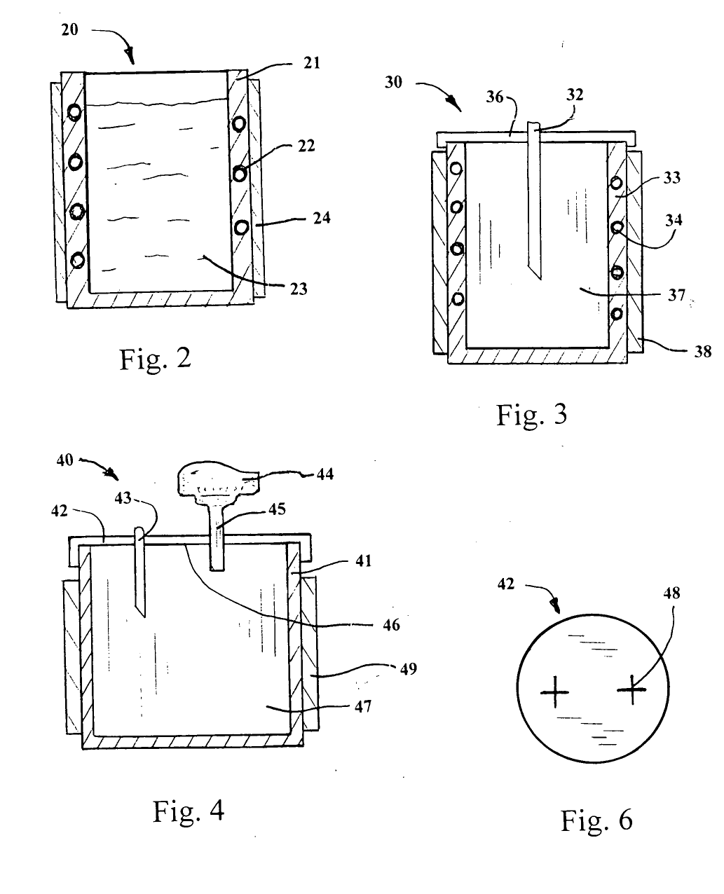

FIG. 2 shows a water receptor for forming a coating of ice on a hypodermic needle or syringe. The receptor and water holder 20 includes a water container 21 that optionally may include refrigeration coils 22 for cooling the water 23 contained in the receptor chamber 21. A standard refrigerant or cryogenic fluid can be passed through the coils. An insulation 24 surrounds the water chamber 21. The water used is preferably sterilized and contains a disinfectant or purifier. The needle to be coated is cooled below the freezing temperature of the water by refrigeration or cryogenic fluid. The water 23 in the container can be room temperature but is preferably cooled to between 35° F. to 40° F. to reduce freezing time.

FIG. 3 shows a refrigeration cooling device 30 for cooling a hypodermic needle 32. The cooler 30 includes a container 33 having an outer insulation 38 and an internal cooling coil 34 shown although the coil could be in the internal chamber 37 and the container could be an insulator. The entrance to the container has an elastic cover 36 through which the needle 32 can be passed into the container for getting cooled. It is preferred that the internal chamber 37 be cooled below 0° F. although it can function reasonably well at temperatures below 20° F.

FIG. 4 is an alternative device for cooling the needle. The device makes up a cryogenic cooler 40 including a cryogenic container 41 with an outer insulating cover 49 and an elastic membrane 42 covering the container opening 46. The membrane 42 accommodates the passage of hypodermic needle 43 into the cryogenic chamber 47 where it is cooled. The membrane 42 also accommodates the passage of a cryogenic discharge 45 for passing cryogenic fluid from a cryogenic fluid dispenser 44 into the chamber 47 and to any needle 43 extending into the chamber 47. The cryogenic fluid may be any of those in common use under pressure or a gas or liquid that on expansion or gasification gets cold. It is preferred that the temperature in the chamber 47 be below −50° F. for rapid cooling.

The hypodermic needle rendered cold in the refrigerant cooling container 30 or the cryogenic cooling chamber 40 can be dipped into the water holder 20 to form an ice coating on the needle. It is preferred that the needle be cooled to below 0° F. although it can be used as long as it is cooled below 20° F.

FIG. 5 shows a freezer 50 having several ice mold cavities 55 in a freezer block 51 for reception of water. The freezer block is provided with refrigerant coils 53. An elastic bladder 56 covers the entrance access opening 57 of the mold cavities 55. A hypodermic needle 52 extends through the elastic bladder. As an alternative the cold cavities can be formed of thin heat conductors 58 with mold cavities 54 and the refrigeration coils 59 wrapped around their outer surface as shown in the central area of FIG. 5. In operation water is poured into the ice mold cavity 55 or mold cavity 54 in the thin conductor 58 after which the elastic bladder is placed over access openings 57. The hypodermic needle is then inserted through the elastic bladder and refrigerant is passed through the refrigerant coils 53 or 59 to freeze the water and coat the needles. The temperature of the ice form should be below 20° F. but preferably is below 0° F.

FIG. 6 is a top view of an elastic bladder 42 through which the needle can be inserted. As an alternative a slit valve can be provided for easy insertion and removal with an ice coating. There is a showing of slit valves 48 of the type that can be used in all the elastic bladders to provide easy access for the hypodermic needles and cryogenic discharge.

It is believed that the construction, operation and advantages of this invention will be apparent to those skilled in the art. It is to be understood that the present disclosure is illustrative only and that changes, variations, substitutions, modifications and equivalents will be readily apparent to one skilled in the art and that such may be made without departing from the spirit of the invention as defined by the following claims.

1. An ice coated elongated hypodermic needle including:
said elongated hypodermic needle having a front end and a rear end;
said hypodermic needle rear end having a syringe attaching connection;
said hypodermic needle front end having a sharp needle tip;
said elongated needle front end having an ice coating.

2. An ice coated elongated hypodermic needle as in claim 1 wherein:
said elongated needle front end and sharp needle tip are both coated with ice.

3. An ice coated elongated hypodermic needle as in claim 2 wherein:
said ice on said elongated needle front end is coated with ice to a thickness of from 0.010 to 0.125 inch.

4. An ice coated elongated hypodermic needle as in claim 2 wherein:
said ice contains a disinfectant.

5. An ice coated elongated hypodermic needle as in claim 4 wherein:
said elongated needle front end terminates in a sharp needle tip that is a flesh penetrating point;
said elongated needle front end and flesh penetrating point are coated with ice.

6. An ice coated elongated hypodermic needle as in claim 1 wherein:
said ice coating includes a disinfectant.

7. An ice coated elongated hypodermic needle as in claim 2 wherein:
said ice coating said hypodermic needle is formed by cooling said hypodermic needle and dipping said cooled hypodermic needle in water.

8. An ice coated elongated hypodermic needle as in claim 7 includes:
cooling said water to between 35° F. to 40° F. before dipping said hypodermic needle into it.

9. An ice coated elongated hypodermic needle as in claim 7 including:
cooling said hypodermic needle by placing it in a refrigeration chamber.

10. An ice coated elongated hypodermic needle as in claim 9 including:
cooling said refrigeration chamber to a temperature below 0° F.

11. An ice coated elongated hypodermic needle as in claim 2 including:
cooling said hypodermic needle to a temperature below 0° F.

12. An ice coated elongated hypodermic needle as in claim 7 including:
cooling said hypodermic needle by placing it into an enclosed chamber;
inserting a cryogenic fluid into said chamber to cool said hypodermic needle.

13. An ice coated elongated hypodermic needle as in claim 12 including:
inserting said cryogenic fluid into said chamber to reduce the temperature within said chamber below −50° F.

14. An ice coated elongated hypodermic needle as in claim 2 including:
placing said hypodermic needle in a water containing mold within a freezer;
freezing said water around said needle by lowering the temperature within said mold.

15. An ice coated elongated hypodermic needle as in claim 14 including;
lowering the temperature within said mold below 0° F.
